(19) United States
(12) Patent Application Publication
(10) Pub. No.: US 2010/0101002 A1
(43) Pub. Date: Apr. 29, 2010

(54) Sock with electronic muscle stimulator integrated, massage socks

(76) Inventor: Roy C. Sanchez, Las Vegas, NV (US)

Correspondence Address: ROY SANCHEZ, 7231 EMPRESS DR., LAS VEGAS, NV, 89147, US

(21) Appl. No.: 12/260,783

(22) Filed: Oct. 29, 2008

(51) Int. Cl. A43B 17/00; A61N 1/00

(52) U.S. Cl. 2/239; 607/144

(57) ABSTRACT

A pair of socks with electronic muscle stimulator device attached to socks to produce electrical stimulants inside socks allow user to massage their feet. Socks that integrate electronic muscle stimulator, conductive fabric, and electrodes woven into socks to distributes electric signals to all parts of socks, gives user the ability to stimulate, contract, massage, and relax feet muscles inside the socks without taking off shoes.

### FIELD OF THE INVENTION

This invention relates generally to therapeutic socks and hosiery accessories and more particularly, describes socks with an electronic muscle stimulator and electrodes integrated into socks to stimulate and massage the feet.

### BACKGROUND OF THE INVENTION

Lots of people work on their feet standing around at their job all day. A few hours into the day of standing and walking around at the job will cause tiredness and fatigue on the feet. Wearing socks and shoes restrict blood flow to the toes, heel, and arch of the feet. Shoe insoles only soften and cushion the inside of the shoe. Shoe insoles do little to alleviate aching feet muscles, pain, and fatigue. Shoe insoles wear out and they don't massage the feet muscles.

Clearly there is a need to massage the feet while wearing socks and shoes. The ability to massage the feet without taking off your shoes and socks allows and individual to find relief at work. Socks that can contract and relax the feet while inside the shoes would get rid of aching muscles, pain, fatigue and pressure. Socks that can massage the feet will be convenient and inconspicuous at work.

Further, such a needed device would be easy to manufacture, use, and maintain allowing massaging of the feet not currently possible with socks. The present invention fulfills these needs and provides further related advantages.

### SUMMARY OF THE INVENTION

The invention provides socks with an electronic muscle stimulator device attached to a conductive fabric containing electrodes integrated into socks to contract and relax feet muscle of a user. An electronic muscle stimulator device is attached to the cuff of the socks which can be turned on and off. The muscle stimulator device sends bioelectrical signals via conductive yarn through out the socks. The integrated electrodes distributes the signals to the muscle of the feet.

The present invention has certain advantages over existing electronic muscle stimulator devices. First the electronic muscle stimulator device is attached to the socks around the opening cuff so the user can reach the on switch. The electronic muscle stimulator device is small, light weight, comfortable to wear. Second the socks have built in conductive yarn and electrodes woven into the fabric. The conductive fabric socks allow the bioelectricity to pass into the skin of the feet. The pulse of the current contract and relax the muscles, relieving aches, sore, and fatigue of the feet. The present invention can be used to relax and massage the feet anywhere the user goes. The sock muscle stimulator has a features no other socks have which allows the users to stimulate their feet in any activity, working, walking, standing up, sitting, or on the go. The electronic muscle stimulator sock is convenient enough to massages the feet in the comfort of your shoes while at work. The present embodiment allows the ability for socks to stimulates the feet by electric pulse to contract and relax the muscle for deep tissue massage which increases endurance and stamina for the feet.

The present invention can be used by any individual because it's easily worn, comfortable to wear, convenient to turn on, and be used any where. The user of the device does not have to worry about turning off the bioelectricity because the muscle stimulator device auto shuts off. The present device stimulates and massages the feet on the go never before possible by a sock. Other features and advantages of the present invention will become apparent from the following detailed description, and accompanying drawings, show the principles of the invention.

### BRIEF DESCRIPTION OF THE DRAWINGS

The accompanying drawings illustrate the invention in such drawings:

FIG. 1 is a perspective illustration of the invention, illustrating a pair of socks with an electronic muscle stimulator device attached to the cuff combine with conductive yarn fabric and electrodes integrated into socks;

FIG. 2 is an exploded perspective view of the invention, illustrating components for the electronic muscle stimulator device, which consist of power button, circuit board, battery, housing, fastener;

FIG. 3 is a partial bottom plane view of the embodiment of the invention, illustrating the bottom surface of the socks where conductive fabric and electrodes are scattered through out the socks allowing bioelectricity signal pulse to contract the feet.

### DESCRIPTION OF THE PREFERRED EMBODIMENT

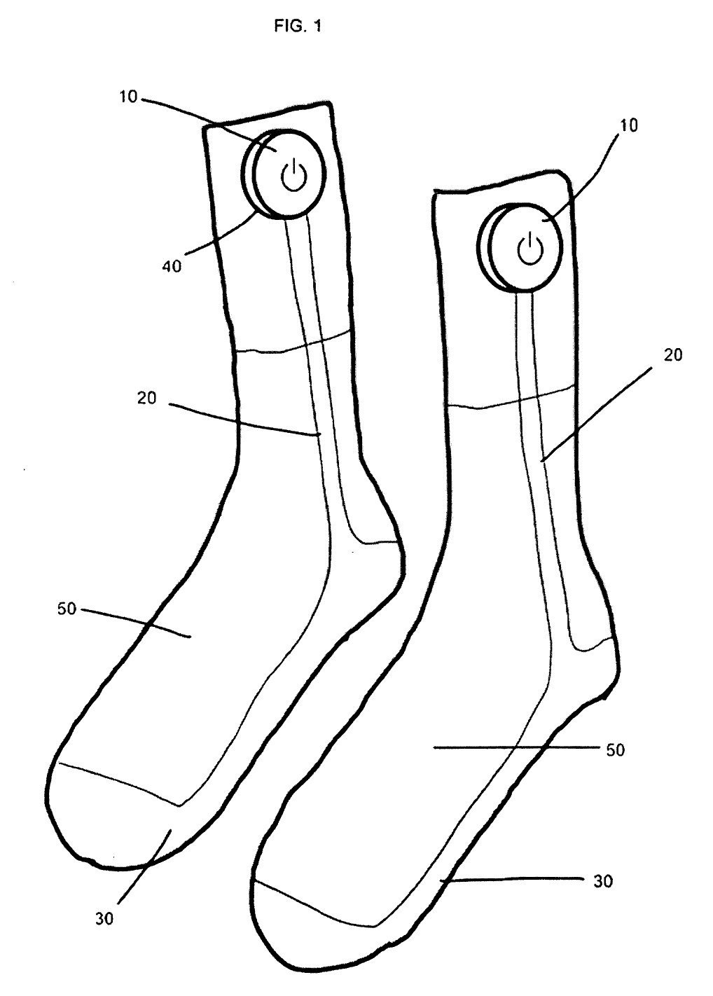

FIG. 1 shows a pair of socks with an electronic muscle stimulator device 10 attached to the top portion of the socks which allows the user to turn on and off the electronic muscle stimulator device to produce bioelectrical signals. The 40 fastener of the muscle stimulator direct contact with the conductive fabric 20 allows the electric signals to travel inside socks 50 down to the bottom area connecting the electrodes 30. The integrated conductive fabric has electrodes 30 woven into the socks and distributes the electric current pulse into the feet.

The electronic muscle stimulator device 10 generates electrical pulses that sends them through conductive yarn fabric 20 to the electrodes 30 and the electric current transfer from electrodes 30 to the skin. The electric current contracts and relaxes the muscles of the feet. There are several electrodes 30 that are scattered in the socks. The muscle stimulator device 10 has a push button on and off switch. The electronic muscle stimulator device 10 encapsulates the circuit board components that operates the stimulator and holds the battery. The battery is easy to maintain and replace. The housing for the battery and circuit board have a water proof gasket. The muscle stimulator device 10 has a secure fastener 40 to attach to the socks. The muscle stimulator device 10 can be detached so the socks can be washed. The muscle stimulator device 10 is convenient to use, comfortable to wear, and easy to maintain.

To operate the muscle stimulator 10, push the on button to generate electric current that travels through out the socks via conductive fabric 20 and electrodes 30. The electrodes 30 distributes the electric pulse current to the feet. The feet reacts by contracting and relaxing the muscles. After several minutes of massaging the feet, the muscle stimulator 10 auto shuts off. The user can manually shut off the muscle stimulator 10 by pushing the off button for a shorter session.

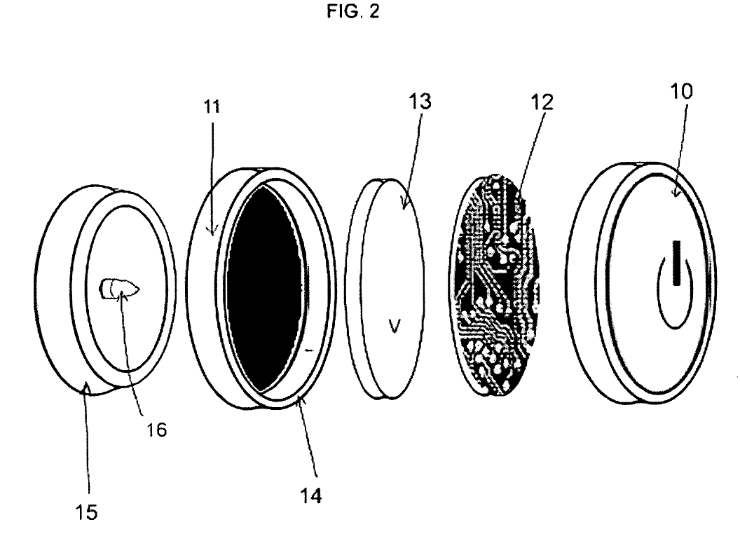

FIG. 2 is a detailed exploded view of the electronic muscle stimulator device showing the assembly of the on/off button 10, circuit board 12, battery supply 13, housing for the components 11, and water proof gasket 14; combine with a fastener 15 containing a conductive path 16 for the electric signals to transfer into the socks. All components are small and light weight so it looks and feels like part of the socks. The brand name can be labeled on the on/off button and a tiny indicator light glows to show the device status is on and working.

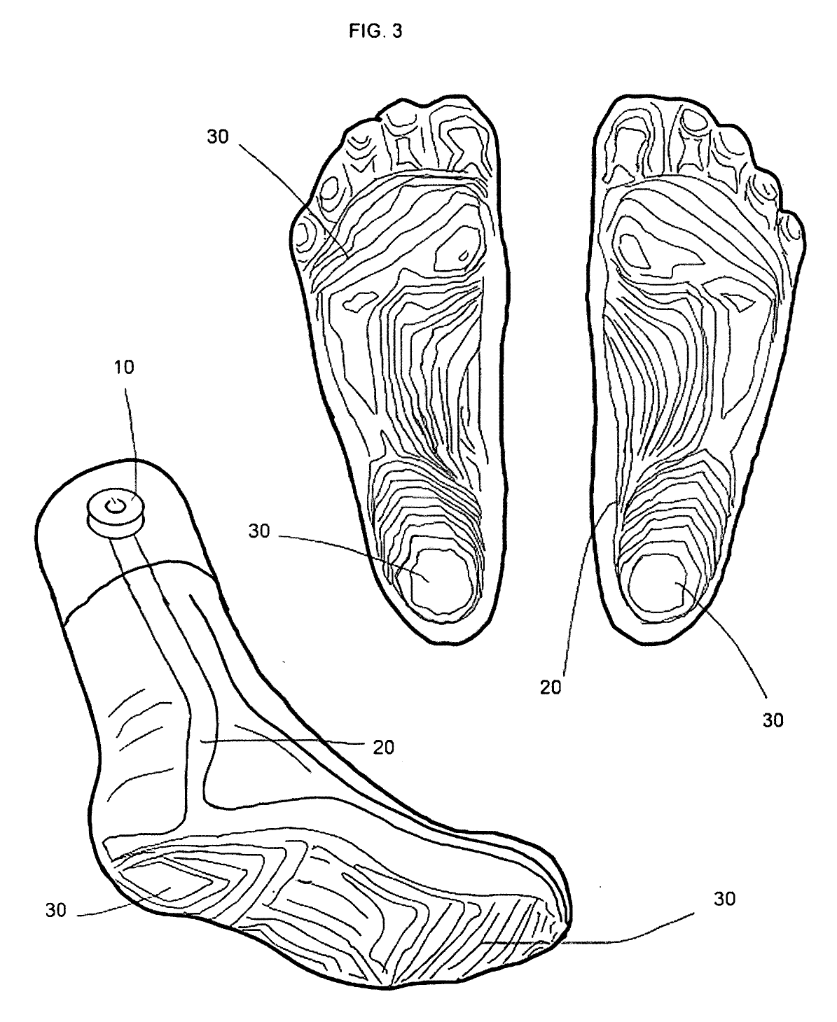

FIG. 3 is a perspective view of the bottom of the socks with conductive fabric 20 and electrodes 30 scattered through out the socks to ensure even distribution of electric signals to massage the feet. Advantage of multiple conductive paths means even if there is a tear in the sock, it won't stop the signals from traveling towards its destination. Redundant path for the electric current allows continuous distribution to other parts of sock, even if there is hindrance and damage to the conductive fabric.

The socks with an electronic muscle stimulator integrated in FIG. 1, is well suited for working people that stand on their feet and walk all day. The electronic muscle stimulator sock FIG. 1 allows the user to stimulate and massage their tired aching feet at work or any activity. The scope of the invention can be adapted to different types of socks, hosiery, stockings, pantyhose, and comes in any size, color, and length. The integration of an electronic muscle stimulator device into socks will be the first of its kind and allow many workers to stimulate and massage their feet while working at their job. The embodiment of the invention is susceptible to various modification and adaptation be interpreted in conjunction with the appended claims.

What is claimed is:

1. Sock that produces electrical current inside the sock allows user to massage and stimulate feet using electrical pulse inside sock comprising:
a) an electronic muscle stimulator device attached to sock;
b) a conductive yarn fabric material and,
c) electrodes woven into sock that carries the electric signal produced by the electronic muscle stimulator device to all parts of sock.

2. Sock with the ability to contract and relaxes feet muscles inside sock using electrical pulse current travels through the conductive electrodes woven into socks and electrical stimulants provide deep tissue massage, to every part of the feet, toes, arch, heel, ankle, and calves; relieves feet of aching muscles, fatigue, soreness, and increase endurance and stamina.

3. Sock with an on and off button on the electronic muscle stimulator device allows the user to turn on and off the electric stimulants in sock by a push of a button; a water proof gasket housing that holds the circuit board and the battery source for the electronic muscle stimulator device which can be open to replace the battery; the electronic muscle stimulator device has an auto shut off feature and can be manually turned off by user.

4. Sock with a fastener that secures the electronic muscle stimulator device to the sock and holds it in place; conductive fastener transfer electrical signal from muscle stimulator device into sock; the muscle stimulator device can be detached from the sock, allowing the user to wash the sock.

5. Sock of claim 4, with an electronic muscle stimulator attached to the cuff of sock allow user wearing socks and shoes, to reach down turn on stimulator device and to generate electrical pulse to provide deep tissue massage inside socks without taking off their shoes; great relief for long hours at work.

6. Sock with seamless integration of conductive fabric and electrodes woven into fabric allows maximum distribution of electric signal to feet even when sock is crooked on foot; conductive fabric have multiple path for electric signal to travel, even if there is a tear or rip in sock, doesn't stop the electrical pulse from reaching destination.

7. Sock integrate with electronic muscle stimulator, electrodes, and conductive fabric which the embodiment can be adapted to any hosiery, socks, stockings, pantyhose, giving a pair of socks the ability to contract and relax feet muscles by electrical stimulants to offer relief without having to take off the shoes; massage socks can be functioning while standing, walking, working; bring therapeutic benefit; it's comfortable to wear, convenient to use, simple to maintain and easy to operate.
